(19) United States
(12) Patent Application Publication
(10) Pub. No.: US 2006/0205753 A1
(43) Pub. Date: Sep. 14, 2006

(54) Use of methylnaltrexone and related compounds to treat post-operative gastrointestinal dysfunction

(76) Inventor: Robert J. Israel, Suffern, NY (US)

Correspondence Address: WOLF GREENFIELD & SACKS, PC, FEDERAL RESERVE PLAZA, 600 ATLANTIC AVENUE, BOSTON, MA, 02210-2206, US

(21) Appl. No.: 11/336,418

(22) Filed: Jan. 20, 2006

Related U.S. Application Data
(60) Provisional application No. 60/645,652, filed on Jan. 20, 2005.

(51) Int. Cl. A61K 31/485

(52) U.S. Cl. 514/282

(57) ABSTRACT

Methods and compositions for treating post-surgical gastrointestinal dysfunction are provided. Methods include administering a quaternary derivative of noroxymorphone (e.g., methylnaltrexone) to a patient after a segmental colectomy is performed on the patient.

### RELATED APPLICATIONS

This application claims benefit under 35 U.S.C. 119(e) of the filing date of U.S. Ser. No. 60/645,652 filed on Jan. 20, 2005, the entire disclosure of which is incorporated herein by reference.

### FIELD OF THE INVENTION

The invention relates to compositions and methods for treating post-operative gastrointestinal dysfunction.

### BACKGROUND OF THE INVENTION

Gastrointestinal dysfunction is a temporary side-effect of abdominal surgery. Post-surgical gastrointestinal dysfunction results from impaired gastrointestinal motility and is characterized by a delayed or reduced gastric emptying, a partial or complete inhibition of intestinal motility (e.g., a partial or complete loss of peristaltic function in at least a part of the intestines), a slowing or complete inhibition of oral-cecal transit, and/or a reduction or absence of Taxation. Post-surgical gastrointestinal dysfunction can cause nausea, vomiting, difficulty or inability to tolerate imbibing liquids or ingesting solids, bloating, gastrointestinal pain, and difficulty or inability to pass gas (flatus) or stool (bowel movement).

Gastrointestinal dysfunction following abdominal surgery is believed to be caused in part by endogenous opioids released during or after the surgical procedure. Exogenous opioids administered to a patient also may contribute to the inhibition of gastrointestinal motility. Gastrointestinal dysfunction following abdominal surgery is temporary and typically lasts for several days. However, it may delay patient discharge from the hospital and can result in clinical complications. In some instances, it may last for up to several weeks and can result in patient readmission to the hospital.

Gastrointestinal dysfunction requiring clinical management following abdominal surgery is typically referred to as post-surgical or post-operative ileus, a period of transient cessation of normal bowel function with a variable reduction in activity sufficient to prevent effective transit of intestinal content. Furthermore, depending on the length of the gastrointestinal dysfunction and the possible recurrence of it, the post-operative ileus can be defined as prolonged post-operative ileus and recurring ileus, respectively. The pathogenesis is contributed to by a complex series of relationships between inhibitory neural reflexes, neurotransmitter and inflammatory mediator release, in addition to the endogenous opioids.

The resolution of post-operative ileus is a gradual process. General opinion suggests that duodenal motility does not stop or stops very briefly following surgery. Gastric motility returns very quickly, usually within 12 hours following surgery. The colonic activity is last to return, usually at least 3-4 days after surgery. Studies using implanted colonic electrodes show the presence of uncoordinated, random bursts of activity, which become more prolonged and progress in an aboral direction with increasing time after surgery. When sufficiently coordinated, after 3-4 days, they are associated with early signs of restored gastrointestinal function such as passage of flatus.

Abdominal surgery also may result in obstructive ileus. Post-surgical obstructive ileus is a gastrointestinal blockage caused by a physical obstruction of the gastrointestinal tract due to the obstructive presence of blood, mucus, sutures, scarring, post-surgical adhesion, or other physical obstruction or lumen narrowing resulting from the surgical procedure. As used herein, gastrointestinal dysfunction is not meant to embrace obstructive ileus.

Several human studies have been performed to evaluate the effectiveness of Alvimopan (Entereg), a piperidine-N-alkylcarboxylate opioid antagonist developed by Adolor Corporation, on post-operative gastrointestinal dysfunction. (Wolff et al., Annals of Surgery, 2004, volume 240, number 4, pp 728-735, Adolor News Release on Dec. 23, 2004, and Adolor Conference Presentations: NewsMakers in the Biotech Industry Investor Conference, Sep. 4, 2003; and 2004 Merrill Lynch Pharmaceutical, Biotechnology & Medical Device Conference, Feb. 3, 2004). Alvimopan is characterized by Adolor Corporation as a mu opioid receptor antagonist with greater affinity and selectivity than methylnaltrexone, and greater potency than methylnaltrexone in antagonizing certain effects of morphine. These studies reported oral administration of Alvimopan starting at least two hours prior to different forms of abdominal surgery (including large and small bowel resection, hysterectomy) to treat different types of diseases (including colon cancer, rectal cancer, Crohn's disease, and uterine cancer). A series of phase III human studies using 6 mg and 12 mg oral doses of Alvimopan produced inconsistent results that lacked statistical significance for many of the end-points being studied.

### SUMMARY OF THE INVENTION

Aspects of the invention relate to restoring gastrointestinal activity in human segmental colectomy patients following surgery. Applicants have demonstrated that gastrointestinal recovery can be accelerated in humans after a segmental colectomy by administering to a patient, after surgery, a quaternary derivative of noroxymorphone (e.g., methylnaltrexone, referred to herein as MNTX) which has a relatively low affinity for mu opioid receptors.

Applicants' discovery is surprising in view of the reports relating to Alvimopan, because Applicants used a relatively low affinity peripheral mu opioid antagonist and Applicants initiated administration of the quaternary derivative of noroxymorphone only after surgery. Applicants' discovery is further surprising, because Applicants were able to accelerate the restoration of gastrointestinal activity in patients after segmental colonic surgery, a type of surgery which could be expected to induce more severe gastrointestinal dysfunction than other forms of surgery (including other abdominal surgeries) that do not involve cutting and suturing the colon. This discovery also is unexpected in view of a rat MNTX study submitted during prosecution of U.S. patent application Ser. No. 10/171,299. This rat study only reported signs of partial recovery of gastrointestinal transit when MNTX was administered intravenously 90 minutes before surgery but not when it was administered closer to surgery (60 minutes or 45 minutes before surgery).

In one aspect, the invention relates to using a quaternary derivative of noroxymorphone to treat post-operative gastrointestinal dysfunction in a patient after a segmental colectomy. According to the invention, the administration of a quaternary derivative of noroxymorphone to a human segmental colectomy patient is initiated post-operatively. In view of the reported studies on Alvimopan, the invention is based, in part, on the unexpected finding that a quaternary derivative of noroxymorphone of relatively low affinity can effectively treat post-surgical gastrointestinal dysfunction in a human segmental colectomy patient when administration is initiated only after surgery. Contrary to the studies in the prior art, this is believed desirable according to the invention in order to maintain the bowel in a quiescent state during the surgery. It was unpredictable based on the prior art whether this approach would work. It also was unpredictable based on the prior art whether the treatment would be sufficient to achieve meaningful clinical endpoints, thereby speeding a patient's recovery and discharge from the hospital.

Accordingly, in some embodiments the administration to a patient of at least one quaternary derivative of noroxymorphone is initiated after a segmental colectomy in order to accelerate the restoration of gastrointestinal activity in the patient (e.g., to accelerate the occurrence of one or more gastrointestinal functions, including a first bowel movement by the patient, patient tolerance of a first full liquid diet, patient tolerance of a first solid meal, patient ingestion of a first solid meal and a first bowel movement by the patient, a first flatus by the patient, gastrointestinal sounds in the colon of the patient). Surprisingly, the treatment accelerates one or more of these functions by clinically significant amounts, that is, by at least 6 hours, 12 hours, 18 hours, 24 hours and even 30 hours, thereby reducing the chances of complications and allowing a patient to be discharged from a hospital a day sooner.

In another aspect, the invention relates to methods and compositions for treating post-operative gastrointestinal dysfunction in a segmental colectomy patient by administering a low affinity peripheral mu opioid receptor antagonist parenterally to the patient post-operatively. Accordingly, in some embodiments the parenteral administration to a human patient of at least one low affinity peripheral mu opioid receptor antagonist is initiated after a segmental colectomy in order to accelerate the restoration of gastrointestinal function in the patient. In one embodiment, the low affinity peripheral mu opioid receptor antagonist, methylnaltrexone (MNTX) is administered parenterally to a segmental colectomy patient, after surgery, in an amount effective to increase the likelihood of shortening the time to a first bowel movement.

According to the invention, a low affinity peripheral mu opioid receptor antagonist is an opioid receptor antagonist with a Ki of between 1 nM and 1 μM (e.g., between about 5 nm and about 100 nM, between about 10 nM and about 90 nM, between about 20 nM and about 80 nM, between about 28 nM and about 68 nM, about 28 nM, etc.) for a mu opioid receptor. In one embodiment, the low affinity peripheral mu opioid receptor antagonist, MNTX is administered intravenously to a segmental colectomy patient in an amount effective to prevent or reduce one or more symptoms of post-operative gastrointestinal dysfunction.

Accordingly, one aspect of the invention provides a method of treating a human patient after a segmental colectomy by initiating post-operatively a parenteral administration of a quaternary derivative of noroxymorphone to a patient after a segmental colectomy. The quaternary derivative of noroxymorphone is administered in an amount sufficient to achieve one or more clinical endpoints as described herein. In one embodiment, the quaternary derivative of noroxymorphone is administered in an amount sufficient to increase the likelihood of shortening the time to a first bowel movement by the patient after the segmental colectomy. In another embodiment, the quaternary derivative of noroxymorphone is administered in an amount sufficient to increase the likelihood of shortening the time to discharge eligibility of the patient after the segmental colectomy. In another embodiment, the quaternary derivative of noroxymorphone is administered in an amount sufficient to increase the likelihood of shortening the time to actual discharge of the patient after the segmental colectomy. In another embodiment, the quaternary derivative of noroxymorphone is administered in an amount sufficient to increase the likelihood of shortening the time to a combination of patient ingestion of a first solid meal and a bowel movement by the patient after the segmental colectomy. In another embodiment, the quaternary derivative of noroxymorphone is administered in an amount sufficient to increase the likelihood of shortening the times to a first bowel movement by the patient after the segmental colectomy; discharge eligibility of the patient after the segmental colectomy; and, a combination of patient ingestion of a first solid meal and a first bowel movement by the patient after the segmental colectomy.

In certain embodiments, the segmental colectomy may be a sigmoidectomy. Alternatively, the segmental colectomy may be a right hemicolectomy, a left hemicolectomy, a transverse colectomy, a colectomy takedown, or a low anterior resection (LAR).

In certain embodiments, the quaternary derivative of noroxymorphone may administered by injection. The injection may be intravenous. The administration is post-operatively that is, the quaternary derivative of noroxymorphone may be initiated less than 7, 6, 5, 4, 3, 2, or even 1 day after surgery. In certain embodiments, it is initiated about 90 minutes after surgery or even immediately after the surgery.

In certain embodiments, the first bowel movement occurs within 5 days after the first administration of the quaternary derivative of noroxymorphone. In other embodiments, the first bowel movement occurs within 4 days, 3.5 days, 3 days, 2.5 days, 2 days or even 1.5 days after the first administration of the quaternary derivative of noroxymorphone. In other embodiments the probability of bowel movement within 5 days, or 4 days, or 3 days, 2 days or even one day is increased.

In certain embodiments, the patient is eligible for discharge within 5 days after the first administration of the quaternary derivative of noroxymorphone. In other embodiments, the patient is eligible for discharge within 4 days, 3 days or 2 days or even one day after the first administration of the quaternary derivative of noroxymorphone.

In certain embodiments, restoration of gastrointestinal activity is indicated by a combination of patient ingestion of a first solid meal and a first bowel movement by the patient within 5 days after the first administration of the quaternary derivative of noroxymorphone. In other embodiments, restoration of gastrointestinal activity is indicated when a combination of the patient ingesting a first solid meal and having a first bowel movement occurs within 4.5 days, 4 days, 3.5 day, 3 days, 2.5 days or even 2 days after the first administration of the quaternary derivative of noroxymorphone.

In certain embodiments, the quaternary derivative of noroxymorphone is administered per dose at about 0.05 to 0.45 mg/kg body weight of the patient. In a preferred embodiment, the dose is intravenous, at 0.3 mg/kg every 6 hours, or 1.2 mg/kg per day. The quaternary derivative of noroxymorphone may be administered between about once per hour and about once per day. The quaternary derivative of noroxymorphone may be administered about once every six hours. The quaternary derivative of noroxymorphone also may be administered repeatedly over a time period of between 1 and 7 days or longer. However, other doses, frequencies and durations of administration may be used as the invention is not limited in this respect.

In certain embodiments, the quaternary derivative of noroxymorphone may be administered at a dose that is less than 50% of the dose at which orthostatic hypotension first appears in humans. In other embodiments, the quaternary derivative of noroxymorphone may be administered at a dose that is less than 50% of the dose at which a lowering of mean arterial blood pressure first appears in humans.

In certain embodiments, a quaternary derivative of noroxymorphone may be administered to the patient orally after a first period of parenteral administration (e.g., 1, 2, 3, 4, 5, 6, 7, or more days after surgery).

Aspects of the invention also may include administering to the patient one or more of an anti-emetic composition, an anti-microbial agent (e.g., an antibiotic or an anti-viral agent). Aspects of the invention also may include administering an opioid to the patient. In one embodiment, a composition comprising a combination of the opioid and the quaternary derivative of noroxymorphone may be administered to the patient. In other aspects of the invention, the patient is receiving morphine (or another opioid administered for pain relief). In one embodiment, the patient may be weaned off morphine (or another opioid administered for pain relief) during a period of time over which the quaternary derivative of noroxymorphone is administered to the patient, whereby the patient receives the quaternary derivative of noroxymorphone even in the absence of administration of or circulating exogenous opioid.

In any of the aspects or embodiments described above, the quaternary derivative of noroxymorphone may be methylnaltrexone. In any of the above embodiments, the treatments may be methods of restoring gastrointestinal activity post surgery. Accordingly, one embodiment of the invention includes a method of restoring gastrointestinal activity in a human patient after a segmental colectomy, by initiating a parenteral administration of methylnaltrexone to a patient after a segmental colectomy, wherein methylnaltrexone is administered in an amount sufficient to restore gastrointestinal activity as indicated by an increase in the likelihood of shortening i) a time to a first bowel movement by the patient after the segmental colectomy; ii) discharge eligibility of the patient after the segmental colectomy; and/or, iii) patient ingestion of a first solid meal and a first bowel movement by the patient after the segmental colectomy. Methylnaltrexone may be infused intravenously. Methylnaltrexone may be administered four times per day at a dose of about 0.3 mg/kg patient weight per administration. Methylnaltrexone may be administered intravenously over a period of 1 to 7 days.

Aspects of the invention include treating post-operative gastrointestinal dysfunction following an abdominal surgery (e.g., a segmental colectomy) that lasts for about 1 to 3 hours, 1 to 4 hours, 1 to 5 hours, 1 to 6 hours, or more or less time. Aspects of the invention may be particularly useful for treating gastrointestinal dysfunction following a segmental colectomy that lasts for less than two hours. The invention provides methods for optimizing the dosage of peripheral mu opioid receptor antagonist to be administered as a function of the duration of the abdominal surgery. In one embodiment, higher amounts of peripheral mu opioid receptor antagonist are administered to a patient for longer surgery times.

Methods and compositions of the invention also are useful to prevent or inhibit (e.g., reduce) the onset of symptoms associated with post-operative gastrointestinal dysfunction. Accordingly, aspects of the invention may be used to prevent or reduce the decrease of one or more gastrointestinal functions in a patient after surgery.

Aspects of the invention also may be used to decrease the amount of time required post-surgery for one or more gastrointestinal functions to be restored (e.g., increased) relative to the amount of time required in the absence of one or more exogenously administered peripheral mu opioid receptor antagonists (typically relative to an amount of time in the presence of a placebo, on average). For example, compositions of the invention may be administered to a segmental colectomy patient to reduce post-operative time(s) to first bowel movement, first flatus, first tolerance of a full liquid diet, first tolerance of a solid diet, recovery, or any combination of two or more of thereof. Aspects of the invention also may be useful to decrease the duration of patient hospital stays after surgery relative to hospital stays in the absence of one or more exogenously administered peripheral mu opioid receptor antagonists. For example, compositions of the invention may be administered to a segmental colectomy patient to reduce post-operative time(s) to eligibility for hospital discharge, actual hospital discharge, or both. Aspects of the invention also may be useful to prevent or reduce patient readmission resulting from post-operative gastrointestinal dysfunction (e.g., due to the recurrence of post-operative gastrointestinal dysfunction).

Aspects of the invention are useful for treating post-operative gastrointestinal dysfunction associated with a segmental colectomy. A segmental colectomy is a surgical removal of only a portion of the colon (e.g., ⅓ of the colon or less), or of a specific region of the colon (e.g., the sigmoid) or a portion thereof (e.g., ⅓ or less), followed by ligation of the remaining gastrointestinal tissue. Colectomies include, but are not limited to, sigmoidectomies, right colectomies, left colectomies, right hemicolectomies, colostomy takedowns, left hemicolectomies, transverse colectomies, appendectomies, and low abdomen resections (LARs).

Aspects of the invention may be useful for treating post-operative gastrointestinal dysfunction associated with a surgical removal of one or more segmental colonic regions associated with a disease. Methods and compositions of the invention may be particularly effective when the disease is a localized disease (e.g., colon cancer, diverticular disease, vascular disease of the colon especially in elderly patients, etc.) as opposed to a disease that affects extended portions of the colon (e.g., inflammatory bowel disease (IBD), Crohn's disease, ulcerative colitis, or other autoimmune disease or inflammatory condition affecting the gastrointestinal tract). Accordingly, aspects of the invention are useful for treating gastrointestinal dysfunction caused by a surgical removal from the colon of one or more polyps, precancerous or cancerous lesions, segmental colonic regions affected by diverticulitis or diverticulosis, or segments of the colon containing one or more polyps, lesions, diseased regions, or a combination thereof.

In aspects of the invention described herein, the peripheral mu opioid receptor antagonist(s) (e.g., quaternary derivatives of noroxymorphone such as MNTX, other peripheral mu opioid receptor antagonists described herein, etc.) may be provided in a pharmaceutically acceptable form (e.g., a form that is acceptable for parenteral administration), and may be administered as a physiologically acceptable preparation (e.g., a sterile physiologically acceptable preparation). One or more peripheral mu opioid receptor antagonists may be administered along with one or more additional pharmaceutical agents. An additional pharmaceutical agent may be an antimicrobial agent (e.g., an antibiotic, an antibacterial agent, or an antiviral agent), an opioid (e.g., morphine), a non-steroidal anti-inflammatory drug (e.g. ketorolac), anti-inflammatory drug, an opiate (e.g. oxycodone) or an anticancer drug. In one aspect, a composition comprising a combination of at least one peripheral mu opioid receptor antagonist and at least one additional pharmaceutical agent may be administered to a patient. Compositions of the invention may be formulated appropriately according to the route of delivery.

Aspects of the invention also include kits. A kit may be a package containing a preparation of at least one peripheral mu opioid receptor antagonist (e.g., a low affinity peripheral mu opioid receptor antagonist, a quaternary derivative of noroxymorphone such as MNTX, other peripheral mu opioid receptor antagonists described herein, etc.) and instructions for administration to a segmental colectomy patient starting after a segmental colectomy. The kit also may include at least one additional pharmaceutical agent (e.g., one or more anti-emetic agents, antimicrobial agents, anti-inflammatory agents, anticancer agents, or any combination thereof). The peripheral mu opioid receptor antagonist(s) and the additional agent(s) may be in the same or different formulations. The kit may include any of the formulations described throughout the specification. The kit also may include an administration device for administering one or more of the preparations. The administration device can be any means useful in administering one of the preparations in the kit, such as a syringe, an enema set, an infusion set, an inhaler, a spray device, a tube, etc.

These and other aspects of the invention will be apparent from the detailed description below.

### BRIEF DESCRIPTION OF THE DRAWINGS

In the drawings:

FIG. 1 is a structural representation of MNTX; and

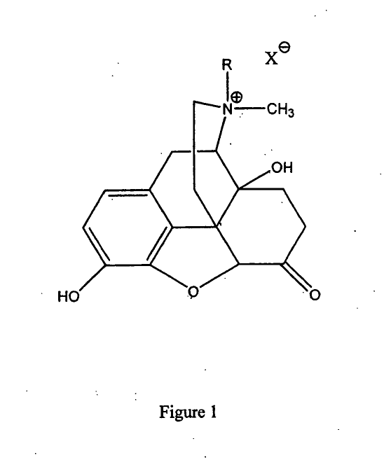

FIG. 2 is a schematic representation of a kit according to the invention.

### DETAILED DESCRIPTION OF THE INVENTION

Recent studies using Alvimopan (a relatively high affinity mu opioid receptor antagonist) have reported using an oral administration at least two hours prior to an abdominal surgery in an attempt to decrease the duration of certain post-operative ileus symptoms. The data from several phase 3 studies using Alvimopan are inconsistent and lack statistical significance with respect to many of the end-points that were evaluated. In this context, Applicants have made the unexpected discovery that MNTX (a peripheral mu opioid receptor antagonist with lower mu opioid receptor affinity than Alvimopan) can significantly reduce the duration of certain symptoms of post-operative gastrointestinal dysfunction following segmental colectomy (via laparotomy) in a human patient when MNTX administration is initiated only after surgery. This discovery also is unexpected in view of a rat MNTX study submitted during prosecution of U.S. patent application Ser. No. 10/171,299. This rat study only reported signs of partial recovery of gastrointestinal transit when MNTX was administered intravenously 90 minutes before surgery but not when it was administered closer to surgery (60 minutes or 45 minutes before surgery).

Accordingly, Applicants have discovered that a segmental colectomy patient is responsive to a quaternary derivative of noroxymorphone (e.g., MNTX) when administered intravenously after surgery. As used herein, a segmental colectomy is a surgical procedure that removes a portion of the colon or a region thereof. A segmental colectomy removes only a part of the colon and not the entire colon. For example, a segmental colectomy may remove about ⅓ or less of the colon, or about ⅓ or less of a specific region of the colon (e.g., the sigmoid). However, the size of the portion or region of the colon that is removed may vary depending on the reason for surgery and the extent of diseased tissue that needs to be removed. Segmental colectomies include, but are not limited to, right colectomies, left colectomies, partial colectomies, transverse colectomies, hemicolectomies (left or right), sigmoidectomies, cecectomies, anterior proctosigmoidectomies, and low anterior proctosigmoidectomies.

Accordingly, one aspect of the invention includes a postoperative parenteral (e.g., intravenous) administration of a quaternary derivative of noroxymorphone (e.g., MNTX) to a patient after a segmental colectomy, with no administration of the quaternary derivative of noroxymorphone prior to surgery or during surgery.

In any aspect of the invention, post-surgical administration of a peripheral mu opioid receptor antagonist may be initiated immediately after surgery, or from minutes (e.g., about 15 minutes, 30 minutes, 45 minutes) to hours (e.g., about 1, 2, 3, etc. hours) to days (e.g., 1, 2, 3, 4, 5, 6, 7 etc.) days after surgery. In one embodiment, a peripheral mu opioid receptor antagonist (e.g., a quaternary derivative of noroxymorphone, MNTX, etc.) is administered starting at 90 minutes after surgery. It should be appreciated that peripheral mu opioid receptor antagonist administration preferably is initiated before a patient recovers gastrointestinal function. In certain embodiments, peripheral mu opioid receptor antagonist administration may only be initiated if a patient has one or more symptoms of gastrointestinal dysfunction lasting for 3 or more days (e.g., administration is initiated at day 4, day 5, or day 6 after surgery if the patient still has one or more symptoms of gastrointestinal dysfunction at that time). However, in other embodiments, one or more doses of a peripheral mu opioid receptor antagonist (e.g., MNTX) may be administered after a patient appears to have recovered sufficient gastrointestinal activity to restore transit of intestinal content and after a bowel movement, in order to prevent or reduce the likelihood of a recurrence of gastrointestinal dysfunction (e.g., after discharge from hospital).

Accordingly, aspects of the invention are useful to treat gastrointestinal dysfunction following a segmental colectomy. The treatment can be to shorten the duration of a post-surgical loss of gastrointestinal motility. The treatment can be to reduce the time to, or accelerate, the first appearance of at least one indicator of restored gastrointestinal motility, including but not limited to, first bowel movement, first flatus, a combination of a first ingestion of a solid meal and a first bowel movement by the patient (solids in-solids out), first tolerance of liquids, first tolerance of a full liquid diet, first tolerance of a solid meal, and recovery of gastrointestinal sounds associated with gastrointestinal motility. Accordingly, the treatment can include accelerating the recovery of upper gastrointestinal function, lower gastrointestinal function, or complete gastrointestinal function after a segmental colectomy. The treatment can also include reducing the time to, or accelerating, patient eligibility for discharge and/or actual patient discharge following a segmental colectomy. Accordingly, aspects of the invention include reducing the length of patient hospital stay following a segmental colectomy. Aspect of the invention also include preventing or reducing the frequency of patient readmission due to gastrointestinal dysfunction following a segmental colectomy.

Aspects of the invention also include restoring gastrointestinal activity after a segmental colectomy. Restoring gastrointestinal activity includes restoring one or more gastrointestinal functions associated with the transit of intestinal content in the colon. Restoring gastrointestinal activity includes reducing the intensity and duration of one or more symptoms of gastrointestinal dysfunction following a segmental colectomy. Restoring gastrointestinal activity includes accelerating the return of one or more gastrointestinal functions after a segmental colectomy.

According to the invention, post-operative gastrointestinal dysfunction is treated by administering a peripheral mu opioid receptor antagonist (e.g., a quaternary derivative of noroxymorphone such as MNTX, other peripheral mu opioid receptor antagonists described herein, etc.) in an amount effective have a clinically significant restorative effect. An effective amount may be an amount that is sufficient to increase the likelihood or probability of restoring one or more functions indicative of gastrointestinal activity in a patient after a segmental colectomy, including but not limited to a first bowel movement by the patient, patient tolerance of a first full liquid diet, patient tolerance of a first solid meal, patient ingestion of a first solid meal and a first bowel movement by the patient, a first flatus by the patient, and gastrointestinal sounds in the colon of the patient. Such amounts are determined for example, as described herein, in clinical studies of patients receiving methylnaltrexone versus those receiving placebo. The amounts, therefore, are based on statistical comparisons of treatment groups and control groups, as expressed in averages or medians or the like. To increase the likelihood or probability in one embodiment, an effective amount may be an amount that is sufficient to accelerate an increase or return of gastrointestinal content transit. An effective amount also may be an amount that is sufficient to increase the likelihood or probability that one or more symptoms of post-surgical gastrointestinal dysfunction are prevented or reduced in a patient after a segmental colectomy. It should be appreciated that different patients may respond differently to treatment due to physiological differences, differences in the type or disease or status, differences in the duration of surgery, differences in the specific region and amount of the colon that is removed. However, as described herein, amounts can be determined that are effective for increasing the likelihood or probability of preventing or inhibiting post-operative gastrointestinal dysfunction or of accelerating the return of gastrointestinal activity in a patient after a segmental colectomy. In addition, the amount administered to an individual patient may be adjusted (e.g., as a function of the status of the patient and the type and duration of surgery) as described herein.

In one embodiment, administration of a quaternary derivative of noroxymorphone (e.g., MNTX) induces a first bowel movement within about 97 hours or about 4 days (on average) after a segmental colectomy. Accordingly, the time to first bowel movement is shortened in this patient population as described below. The first bowel movement was accelerated by about 23 hours or about 1 day (on average) relative to the time in the absence of the quaternary derivative of noroxymorphone.

In one embodiment, administration of a quaternary derivative of noroxymorphone (e.g., MNTX) allows a patient to be eligible for discharge within about 119 hours or about 5 days (on average) after a segmental colectomy. Accordingly, the time to patient discharge eligibility is shortened in this patient population as described below. The time to patient discharge eligibility was accelerated by about 30 hours or by about 1-2 days (on average) relative to the time in the absence of the quaternary derivative of noroxymorphone.

In one embodiment, administration of a quaternary derivative of noroxymorphone (e.g., MNTX) allows a patient to be discharged within about 140 hours or about 6 days (on average) after a segmental colectomy. Accordingly, the time to actual patient discharge is shortened in this patient population as described below. The time to patient discharge was accelerated by about 25 hours or about 1-2 (on average) days relative to the time in the absence of the quaternary derivative of noroxymorphone.

In one embodiment, administration of a quaternary derivative of noroxymorphone (e.g., MNTX) induces a first flatus within about 88 hours or 4 days (on average) after a segmental colectomy. Accordingly, the time to first flatus is shortened in this patient population as described below. The time to first flatus was accelerated by about 8 hours or about half (on average) a day relative to the time in the absence of the quaternary derivative of noroxymorphone.

In one embodiment, administration of a quaternary derivative of noroxymorphone (e.g., MNTX) allows a patient to eat a first solid meal and have a bowel movement (solids in-solids out) within about 124 hours or about 5-6 days (on average) after a segmental colectomy. Accordingly, the time to a combination of a first solid meal and a bowel movement is shortened in this patient population as described below. The time to a first solid meal and a bowel movement was accelerated by about 27 hours or about 1-2 days (on average) relative to the time in the absence of the quaternary derivative of noroxymorphone.

In one embodiment, administration of a quaternary derivative of noroxymorphone (e.g., MNTX) allows a patient to drink a first full liquid diet within about 70 hours or about 3 days (on average) after a segmental colectomy. Accordingly, the time to a first full liquid diet is shortened in this patient population as described below. The time to a first full liquid diet was accelerated by about 30 hours or about 1-2 days (on average) relative to the time in the absence of the quaternary derivative of noroxymorphone.

In one embodiment, administration of a quaternary derivative of noroxymorphone (e.g., MNTX) allows a patient to eat a first solid meal within about 100 hours or about 5 days (on average) after a segmental colectomy. Accordingly, the time to a first solid meal is shortened in this patient population as described below. The time to a first solid meal was accelerated by about 25 hours or about 1 day (on average) relative to the time in the absence of the quaternary derivative of noroxymorphone.

In one embodiment, administration of a quaternary derivative of noroxymorphone (e.g., MNTX) allows a patient to delay a first use of an anti-emetic for about 26 hours or about 1-2 days (on average) after a segmental colectomy. Accordingly, the time to first use of an anti-emetic is lengthened in this patient population as described below. The time to first anti-emetic use was delayed by about 10 hours or half a day (on average) relative to the time in the absence of the quaternary derivative of noroxymorphone. It should be appreciated that in some embodiments a patient does not use an anti-emetic.

In one embodiment, administration of a quaternary derivative of noroxymorphone (e.g., MNTX) induces a first bowel movement within about 94 hours or about 4 days (on average) after a sigmoidectomy. Accordingly, the time to first bowel movement is shortened in this patient population as described below. The first bowel movement is accelerated by about 23 hours or about 1-2 days (on average) relative to the time in the absence of the quaternary derivative of noroxymorphone.

In one embodiment, administration of a quaternary derivative of noroxymorphone (e.g., MNTX) allows a patient to be eligible for discharge within about 105 hours or about 4-5 days (on average) after a sigmoidectomy. Accordingly, the time to patient discharge eligibility is shortened in this patient population as described below. The time to patient discharge eligibility was accelerated by about 40 hours or about 2 days (on average) relative to the time in the absence of the quaternary derivative of noroxymorphone.

In one embodiment, administration of a quaternary derivative of noroxymorphone (e.g., MNTX) allows a patient to be discharged within 125 hours or 5-6 days (on average) after a sigmoidectomy. Accordingly, the time to actual patient discharge is shortened in this patient population as described below. The time to patient discharge was accelerated by about 30 hours or about 1-2 days (on average) relative to the time in the absence of the quaternary derivative of noroxymorphone.

In one embodiment, administration of a quaternary derivative of noroxymorphone (e.g., MNTX) induces a first flatus within about 85 hours or about 3-4 days (on average) after a sigmoidectomy. Accordingly, the time to first flatus is shortened in this patient population as described below. The time to first flatus was accelerated by about 25 hours or about one day (on average) relative to the time in the absence of the quaternary derivative of noroxymorphone.

In one embodiment, administration of a quaternary derivative of noroxymorphone (e.g., MNTX) allows a patient to eat a first solid meal within about 93 hours or about 3-4 days after a sigmoidectomy. Accordingly, the time to a first solid meal is shortened in this patient population as described below. The time to a first solid meal was accelerated by about 29 hours or about one day relative to the time in the absence of the quaternary derivative of noroxymorphone.

Aspects of the invention are useful to prevent or reduce the occurrence of gastrointestinal dysfunction in patients after segmental colectomy. Aspects of the invention also are useful to help patients recover gastrointestinal function after a segmental colectomy. Patients include, but are not limited to, patients who have undergone a partial colectomy, a transverse colectomy, a hemicolectomy (left or right), a sigmoidectomy, a cecectomy, an anterior proctosigmoidectomy, or a low anterior proctosigmoidectomy. In one embodiment, patients include those that have undergone a segmental colectomy via laparotomy with general anesthesia. In another embodiment, patients include those that have undergone a segmental colectomy via laparoscopy. A post-operative patient may have, or be at risk or having, post-operative gastrointestinal dysfunction.

Aspects of the invention may be particularly useful for treating post-operative gastrointestinal dysfunction associated with a surgical removal of one or more gastrointestinal regions associated with a localized disease (e.g., colon cancer, diverticular disease, vascular disease of the colon especially in elderly patients, etc.) as opposed to surgical removal of the entire colon or small intestine as may be required in a disease that affects extended portions of the gastrointestinal tract (e.g., inflammatory bowel disease (IBD), Crohn's disease, ulcerative colitis, or other autoimmune disease or inflammatory condition affecting the gastrointestinal tract). Accordingly, aspects of the invention are useful for treating gastrointestinal dysfunction caused by a surgical removal from the colon of one or more polyps, precancerous or cancerous lesions, regions of the colon or rectum affected by diverticulitis or diverticulosis or vascular disease, or segments of the colon or rectum containing one or more polyps, lesions, diseased regions, or a combination thereof. However, in instances where only a segment of the colon is removed to treat an inflammatory or autoimmune disease or condition, it is expected that methods of the invention will be useful to restore gastrointestinal activity post-operatively as described herein.

Aspects of the invention may be useful for treating post-operative gastrointestinal dysfunction associated with a segmental colectomy surgery that lasted less than one hour, less than two hours (e.g., from about one to two hours), less than 3 hours, less than 4 hours, less than 5 hours, less than 6 hours or more than 8 hours. In other aspects, the invention provides methods for optimizing the dosage of peripheral mu opioid receptor antagonist as a function of the duration of the abdominal surgery. In one embodiment, higher amounts of peripheral mu opioid receptor antagonist are administered to a patient as the duration of the surgery increases.

It is believed that the methods described herein also are useful to restore gastrointestinal activity in patients after sigmoid rectal and rectal surgery. Accordingly, similar clinically significant outcomes are expected to be obtained when treating patients (e.g., accelerating the restoration of one or more gastrointestinal functions indicative of gastrointestinal activity) after sigmoid rectal and rectal surgeries according to the methods described herein for segmental colectomy.

Aspects of the invention relate to administering one or more quaternary derivatives of noroxymorphone to a segmental colectomy patient. A particularly preferred quaternary derivative of noroxymorphone is methylnaltrexone and salts thereof, described first by Goldberg, et al. Methylnaltrexone is also described in U.S. Pat. Nos. 4,719,215; 4,861,781; 5,102,887; 6,274,591; U.S. Patent Application Nos. 2002/0028825 and 2003/0022909; and PCT publication Nos. WO 99/22737 and WO 98/25613; each hereby incorporated by reference. As used herein, “methylnaltrexone” or “MNTX” includes N-methylnaltrexone and salts thereof. Salts include, but are not limited to, bromide salts, chloride salts, iodide salts, carbonate salts, and sulfate salts.

Methylnaltrexone is provided as a white crystalline powder freely soluble in water. Its melting point is 254-256° C. Methylnaltrexone is available in a powder form from Mallinckrodt Pharmaceuticals, St. Louis, Mo. The compound as provided is 99.4% pure by reverse phase HPLC, and contains less than 0.011% unquaternized naltrexone by the same method. Methylnaltrexone is also identified as N-methyl-naltrexone bromide, naltrexone methobromide, N-methylnaltrexone, MNTX, SC-37359, MRZ-2663-BR, and N-cyclopropylmethylnoroxy-morphine-methobromide.

However, aspects of the invention also include administering one or more other peripheral mu opioid receptor antagonists to a segmental colectomy patient. Peripheral mu opioid receptor antagonists are well-known in the art. Peripheral mu opioid receptor antagonists, as used herein, means those which do not effectively cross the blood-brain barrier into the central nervous system. The majority of currently known opioid antagonists act both centrally and peripherally, and have potential for centrally mediated, undesirable side-effects. Naloxone and naltrexone are examples. The present invention involves the art recognized group of compounds known as peripheral mu opioid receptor antagonists.

In preferred form, methods of the present invention involve parenterally administering to a patient, after a segmental colectomy, a compound which is a peripheral mu opioid receptor antagonist compound. The term peripheral designates that the compound acts primarily on physiological systems and components external to the central nervous system, i.e., the compound does not readily cross the blood-brain barrier. The peripheral mu opioid receptor antagonist compounds employed in the methods of the present invention typically exhibit high levels of activity with respect to gastrointestinal tissue, while exhibiting reduced, and preferably substantially no, central nervous system (CNS) activity. The term “substantially no CNS activity”, as used herein, means that less than about 20% of the pharmacological activity of the peripheral mu opioid receptor antagonist compounds employed in the present methods is exhibited in the CNS. In preferred embodiments, the peripheral mu opioid receptor antagonist compounds employed in the present methods exhibit less than about 5% of their pharmacological activity in the CNS, with about 1% or less (i.e., no CNS activity) being still more preferred.

The peripheral mu opioid receptor antagonist preferably has a receptor affinity similar to that of methylnaltrexone. However, it is believed that the unexpected findings of the invention can be extended to a host of peripheral mu opioid receptor antagonists, provided administration is parenteral, is after surgery and provided the surgery is a segmental colectomy. The peripheral mu opioid receptor antagonist may be, for example, a piperidine N-alkylcarboxylate such as described in U.S. Pat. Nos. 5,250,542; 5,434,171; 5,159,081; 5,270,328; and 6,469,030. It also may be an opium alkaloid derivative such as described in U.S. Pat. Nos. 4,730,048; 4,806,556; and 6,469,030. Other peripheral mu opioid receptor antagonists include quaternary benzomorphan compounds such as described in U.S. Pat. Nos. 3,723,440 and 6,469,030. The preferred antagonists are quaternary derivatives of noroxymorphone such as methylnaltrexone, described in U.S. Pat. Nos. 4,176,186 and 5,972,954. Other examples of quaternary derivatives of noroxymorphone include methylnaloxone and methylnalorphine.

In some aspects of the invention, a low affinity peripheral mu opioid receptor antagonist is used. According to the invention, a low affinity peripheral mu opioid receptor antagonist has a lower affinity for a mu opioid receptor than Almivopan (e.g., about 5 fold lower, about 10 fold lower, about 20 fold lower, about 50 fold lower, about 100 fold lower, or more than 100 fold lower, including intermediate values). According to aspects of the invention, a low affinity peripheral mu opioid has a Ki greater than 1 nM (e.g., between 1 nM and 1 μM, between 5 nM and 100 nM, between 5 nM and 50 nM, between 50 nM and 100 nM, between 10 nM and 90 nM, between 20 nM and 80 nM, between 28 nM and 68 nM, about 28 nM, etc.) for a mu opioid receptor. The affinity (e.g. Ki or relative affinity) may be measured using the techniques described in Mitch et al., J Med. Chem. 1993 Oct. 1;36(20):2842-50 and/or Wang et al., FEBS Lett. 1994 Jan. 31;338(2):217-22, the disclosures of which are incorporated by reference herein in their entirety.

A variety of administration routes are available. The particular mode selected will depend, of course, upon the particular combination of drugs selected, the severity of the condition being treated, or prevented, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects.

Parenteral modes of administration include intravenous, subcutaneous, and intramuscular administration. Parenteral modes of administration include injection. As used herein, injection includes infusion. Infusion periods may range from several minutes (e.g., about 5, 10, 15, 20, 25, 30, or more minutes) to several hours (e.g., about 1, 2, 3, 4, 5, or more hours). For continuous infusion, a patient-controlled analgesia (PCA) device may be employed.

However, it should be appreciated that aspects of the invention may include other modes of administration. Other routes of administration may include rectal, topical, transdermal, sublingual, pulmonary, intracavity, aerosol, aural (e.g., via eardrops), intranasal, inhalation, needle less injection, or intradermal (e.g., transdermal) delivery.

In one embodiment, after an initial first parenteral administration following surgery other routes of administration may be used (including oral and other parenteral routes), especially if continued administration to the patient is suggested for a period after recovery of gastrointestinal activity or after patient discharge. Accordingly, oral administration may be initiated after an initial period of post-operative parenteral administration. Oral, rectal, or subcutaneous administration may be important for prophylactic treatment of recurrence or long-term treatment. Preferred rectal modes of delivery include administration as a suppository or enema wash.

Generally, parenteral administration, including intravenous and subcutaneous administration, may be from about 0.001 to 1.0 mg/kg body weight. It is expected that doses ranging from 0.05 to 0.45 mg/kg body weight of a quaternary derivative of noroxymorphone administered by injection will yield the desired results, and doses of 0.1 to 0.3 are preferred. The preferred dose for methylnaltrexone is 0.3 mg/kg in a volume of 20 mg/ml.

Generally, oral doses of the quaternary derivatives of noroxymorphone will be from about 0.25 to about 5.0 mg/kg body weight per day. It is expected that oral doses in the range from 0.5 to 5.0 mg/kg body weight will yield the desired results.

Dosage may be adjusted appropriately to achieve desired drug levels, local or systemic, depending on the mode of administration. For example, it is expected that the dosage for oral administration of the peripheral mu opioid receptor antagonists in an enterically-coated formulation would be lower than in an immediate release oral formulation. In the event that the response in a patient is insufficient of such doses, even higher doses (or effectively higher dosage by a different, more localized delivery route) may be employed to the extent that the patient tolerance permits. Multiple doses per day are contemplated to achieve appropriate systemic levels of compounds. Appropriate system levels can be determined by, for example, measurement of the patient's peak or sustained plasma level of the drug. “Dose” and “dosage” are used interchangeably herein. A dose of a peripheral mu opioid receptor antagonist (e.g., a quaternary derivative of noroxymorphone such as MNTX, etc.) may be administered according to a regular schedule including, but not limited to, hourly, several times a day (e.g., 2, 3, 4, 5, 6, or more times a day), or daily. However, the frequency of administration will be a function of the dose administered and the clinical symptoms of the patient. It should be appreciated that irregular dosing schedules and different doses may be used. For example, the amount administered may be decreased over time (e.g., each successive administration may include a lower dose than the previous one).

One or more peripheral mu opioid receptor antagonists as described herein may be provided in a pharmaceutically acceptable form, and may be administered as a physiologically acceptable preparation (e.g., a sterile pharmaceutical preparation). One or more opioid receptor antagonists may be administered along with one or more additional pharmaceutical agents. An additional pharmaceutical agent may be an antimicrobial agent (e.g., an antibiotic, an antibacterial agent, or an antiviral agent), a pain killer, an opioid (e.g., alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and tramadol), an anti-inflammatory agent, or an anticancer drug. Opioids, antibiotics, antivirals, antibacterials, anti-inflammatories and anticancer agents are described in US 20040266806, US 20040259899, US 20030065023, WO 2004/010998, WO 2004/10997. In one aspect, a composition comprising a combination of at least one opioid receptor antagonist and at least one additional pharmaceutical agent may be administered to a patient. Compositions of the invention may be formulated appropriately according to the route of delivery. Accordingly, a pharmaceutical preparation of the invention may include one or more peripheral mu opioid receptor antagonists along with one or more additional pharmaceutical agents.

Pharmaceutical preparations of the invention, when used in alone or in cocktails, may be administered in therapeutically effective amounts. A therapeutically effective amount will be determined by the parameters discussed herein; but, in any event, is that amount which establishes a level of the drug(s) effective for treating a subject, such as a human subject, having one of the conditions described herein.

In the case of post operative gastrointestinal dysfunction, an effective amount, for example, is that amount which achieves one of the clinical endpoints as described herein.

When administered to a subject, effective amounts will depend, of course, on the particular surgery; the severity of the gastrointestinal dysfunction; individual patient parameters including age, physical condition, size and weight; concurrent treatment; frequency of treatment; and the mode of administration. These factors are well known to those of ordinary skill in the art and can be addressed with no more than routine experimentation.

Pharmaceutical preparations may conveniently be presented in unit dosage form and may be prepared by any of the methods well known in the art of pharmacy. All methods include the step of bringing the compounds of the invention into association with a carrier which constitutes one or more accessory ingredients. In general, the compositions are prepared by uniformly and intimately bringing the compounds of the invention into association with a liquid carrier, a finely divided solid carrier, or both, and then, if necessary, shaping the product.

When administered, the pharmaceutical preparations of the invention are applied in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

The pharmaceutical preparations of the present invention may include or be diluted into a pharmaceutically-acceptable carrier. The term “pharmaceutically-acceptable carrier” as used herein means one or more compatible solid or liquid fillers, diluents or encapsulating substances which are suitable for administration to a human. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

Aqueous formulations may include a chelating agent, a buffering agent, an anti-oxidant and, optionally, an isotonicity agent, preferably pH adjusted to between 3.0 and 3.5. Preferred such formulations that are stable to autoclaving and long term storage are described in application Ser. No. 10/821,811, now published as 20040266806, entitled “Pharmaceutical Formulation,” the disclosure of which is incorporated herein by reference.

Chelating agents include: ethylenediaminetetraacetic acid (EDTA) and derivatives thereof, citric acid and derivatives thereof, niacinamide and derivatives thereof, sodium desoxycholate and derivatives thereof, and L-glutamic acid, N,N-diacetic acid and derivatives thereof.

Buffering agents include those selected from the group consisting of citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid.

Antioxidants include those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollic acid, sodium formaldehyde sulfoxylate, tocopheral and derivatives thereof, monothioglycerol, and sodium sulfite. The preferred antioxidant is monothioglycerol.

Isotonicity agents include those selected from the group consisting of sodium chloride, mannitol, lactose, dextrose, glycerol, and sorbitol.

Preservatives that can be used with the present compositions include benzyl alcohol, parabens, thimerosal, chlorobutanol and preferably benzalkonium chloride. Typically, the preservative will be present in a composition in a concentration of up to about 2% by weight. The exact concentration of the preservative, however, will vary depending upon the intended use and can be easily ascertained by one skilled in the art.

In one embodiment, the formulation is a lyophilized form, for example in a cryo-preservative such as mannitol, lactose, sucrose, and others as disclosed in the published US Patent Application No. 20040266806.

The formulations can be constructed and arranged to create steady state plasma levels. Steady state plasma concentrations can be measured using HPLC techniques, as are known to those of skill in the art. Steady state is achieved when the rate of drug availability is equal to the rate of drug elimination from the circulation. In typical therapeutic settings, the quaternary derivatives of noroxymorphone will be administered to patients either on a periodic dosing regimen or with a constant infusion regimen. The concentration of drug in the plasma will tend to rise immediately after the onset of administration and will tend to fall over time as the drug is eliminated from the circulation by means of distribution into cells and tissues, by metabolism, or by excretion. Steady state will obtain when the mean drug concentration remains constant over time. In the case of intermittent dosing, the pattern of the drug concentration cycle is repeated identically in each interval between doses with the mean concentration remaining constant. In the case of constant infusion, the mean drug concentration will remain constant with very little oscillation. The achievement of steady state is determined by means of measuring the concentration of drug in plasma over at least one cycle of dosing such that one can verify that the cycle is being repeated identically from dose to dose. Typically, in an intermittent dosing regimen, maintenance of steady state can be verified by determining drug concentrations at the consecutive troughs of a cycle, just prior to administration of another dose. In a constant infusion regimen where oscillation in the concentration is low, steady state can be verified by any two consecutive measurements of drug concentration.

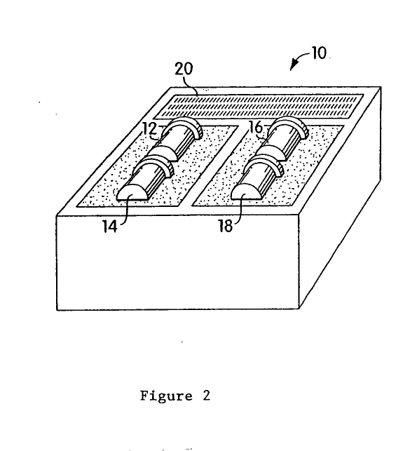

Aspects of the invention also include kits (as shown on FIG. 2). A kit may be a package 10 containing a preparation of at least one peripheral mu opioid receptor antagonist 12, 14, 16, and/or 18 as described herein and instructions for administration 20 to a segmental colectomy patient only after surgery or also just before or at the time of surgery. The kit 10 also may include at least one additional pharmaceutical agent 12, 14, 16, and/or 18. The peripheral mu opioid receptor antagonist(s) and the additional agent(s) may be in the same or different formulations. The kit may include any of the formulations described throughout the specification. The kit also may include an administration device for administering one or more of the preparations. The administration device can be any means useful in administering one of the preparations in the kit, such as a syringe, an enema set, an infusion set, an inhaler, a spray device, a tube, etc.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

The following non-limiting examples relate to a phase 2 human study using MNTX to reduce the duration of gastrointestinal dysfunction following segmental colectomy. The primary objective of this study was to assess the activity of parenterally administered MNTX every six hours compared with placebo in shortening the duration of or preventing post-operative ileus in patients who have undergone segmental colectomies. Evidence of activity of MNTX was based on at least one or more of the following: the time to tolerance of liquids, time to first bowel movement, time to tolerance of solid foods, time to the combination of first solid meal and bowel movement, time to first micturition post foley catheter removal, and time to hospital discharge. The use of daily anti-emetic and opioid medication was also assessed.

The secondary objective of this study was to assess the safety of parenterally administered MNTX every six hours compared to placebo, as measured by adverse events, changes in verbal numerical scales, changes in vital signs, physical exam assessments, the incidence of infections, and changes in laboratory values. In addition, the incidence and severity of nausea and vomiting, pruritus, urinary retention, was evaluated.

A total of 65 patients at eight surgical centers participated in the phase 2 randomized, double-blind, placebo-controlled study. Subjects underwent segmental colectomies primarily due to cancer or diverticular disease. Shortly after surgery (90 minutes post surgery), study medication (0.3 mg/kg MNTX or placebo) was administered intravenously at six-hour intervals for a maximum of seven days.

The following examples are not limiting. However, aspects of the invention described above may incorporate specific compositions, procedural steps, criteria, etc. that are described in the following examples.

### Example 1

### Study Design

The study was a double-blind, randomized parallel group study designed to evaluate the safety and activity of IV (intravenous) MNTX in the treatment of patients who are undergoing segmental colectomies via laparotomy and the duration of their post-operative ileus. Patients were randomized to either placebo (saline) or a fixed dose of IV MNTX of 0.30 mg/kg patient body weight in 50 cc of 0.9% normal saline hung as an IV piggyback (IVPB) to the main line and infused over twenty (20) minutes every six (6) hours until twenty-four (24) hours after the patient could tolerate solid foods, until discharged from the hospital, or for a maximum of seven (7) days. These time points for study drug discontinuation were selected in order to analyze the secondary efficacy endpoints of IV MNTX as well as the primary endpoints.

65 patients were enrolled at approximately 8 study centers. This proposed sample size has approximately 80% power to detect a difference between treatment groups at the 0.05 level of significance.

The following criteria were used to include or exclude patients for the study:

Inclusion Criteria:
Patients must be ≧18 years of age.
Patients must meet the American Society of Anesthesiologists (ASA) physical status I, II, or III.
Patients must have undergone a segmental colectomy. Acceptable procedures include: partial colectomy, colectomy (right or left), transverse colectomy, hemicolectomy (left or right), sigmoidectomy, cecectomy, anterior proctosigmoidectomy, and low anterior proctosigmoidectomy via laparotomy with general anesthesia.
Patients must be receiving opioids via intravenous (IV) patient-controlled analgesia (PCA) for post-operative pain relief.
Patients must have stable vital signs.
Patient must sign an informed consent form.
Females of childbearing potential must have a negative pregnancy test (urine or serum) and must use appropriate forms of birth control (oral, implantable, or injectable contraceptives; spermicide in conjunction with a barrier such as a condom or diaphragm; intrauterine device or IUD) throughout the study.

Exclusion Criteria.
Patients with known hypersensitivity to methylnaltrexone, naltrexone, or naloxone.
Patients who received any investigational new drug (experimental) in the previous 30 days.
Patients with a recent history (≦one year) of abdominal radiation therapy.
Patients with a history of treatment with vinca alkaloids.
Patients undergoing operations for complications related to active inflammatory bowel disease.
Patients undergoing operations resulting in gastrointestinal ostomies.
Patients with a significant medical history and/or any intra-operative findings that might complicate their post-operative course.
Patients receiving spinal/epidural medication for post-operative pain relief.
Patients with a QT_{c} interval greater than 450 ms males or 470 ms females based on the 12-lead screening electrocardiogram (ECG).

The surgical procedures that were performed included right hemicolectomies, colostomy takedowns, sigmoidectomies, left hemicolectomies, transverse colectomies, and LARs.

### Example 2

### Patient Treatment

Screening (Up to 2 Weeks Prior to Surgery). All patients who met the eligibility criteria had the study explained to them, and they signed an informed consent form at their pre-operative visit. A physical examination including vital signs, a detailed medical history, laboratory testing (Chemistry panel and complete blood count (CBC) with diff and platelets), a 12-lead supine, resting ECG, and a review of all past and current medications were obtained on all patients. Females of child bearing potential had a negative urine pregnancy test at least two (2) days prior to dosing.

Day of Surgery. Within twenty-four (24) hours of surgery, patients were randomly assigned to either the MNTX 0.30 mg/kg or placebo (saline) dose group.

Pre-induction management. Upon arrival to the pre-induction area or the operating room, all patients had IV catheters inserted. After induction, all patients received between 7-10 ml/kg of intravenous fluid per hour.

Anesthetic management. Before induction of anesthesia, patients received midazolam (Versed) 0.02-0.04 mg/kg and fentanyl 1.0-3.0 mcg/kg intravenously. Patients were oxygenated and anesthesia was induced and maintained using standard procedures.

Post-operative management. Patients were admitted to the PACU immediately post-op for recovery and analgesic therapy via IV PCA. The IV PCA contained fentanyl, morphine, or hydromorphone. No matter which opioid was used post-operatively the attending physician was responsible for the patient's pain management and was titrating opioids to the patient's optimal comfort. A daily record of the patient's total opioid medication dose was recorded in the case report form.

Prior to the first study drug administration the patient's vital signs were monitored. Patients were asked to assess their level of nausea, abdominal cramping, pain, and itching based on a verbal numerical scale. Within 90 minutes from the end of the surgical procedure, defined as the surgical end time recorded on the operating room (OR) sheet (regardless of whether a patient was in the PACU or not) the first dose of study drug was administered intravenously through an already existing IV line, hung as an IV piggyback (IVPB) to the main line, over 20 minutes. If the IV line was being used for other medications, i.e. antibiotics, electrolyte supplements, etc., the line was flushed with 20 cc of the main IV fluid prior to the hanging of study product. The patient's vital signs were monitored at the end of the study medication infusion, 20 min, 60 min, 120 min and 180 minutes after this first IV study drug administration. Patients were asked to assess their level of nausea, abdominal cramping, pain, and itching based on a verbal numerical scale and whether or not they had vomited 60 minutes after the end of the first study drug administration.

The second dose of study drug was administered six (6) hours after the first dose of study medication.

The patients continued to receive the study product every six (6) hours until twenty-four (24) hours after the patient could tolerate solid foods, was discharged from the hospital, or for a maximum of seven (7) days.

The patients were carefully monitored for any adverse effects related to the study drug throughout the study but especially after the first and second administrations.

Other assessments occurred daily at 7 am, 12 pm, 5 pm, and 10 pm, ±30 minutes, regardless of study drug administration, until twenty-four (24) hours after the patient could tolerate solid foods, was discharged from the hospital, or for a maximum of seven (7) days. The other assessments included:

Verbal scale for nausea, abdominal
cramping, pain, and itching
Vomiting assessment
30 cc liquid challenge
Full liquid and solid diet
advancement
Discharge eligibility criteria
assessment
Bowel sounds auscultation
Patient diary tracking
Adverse event evaluation
Concomitant medication
assessment
Bowel movement
Actual discharge

These daily assessments began on the day of surgery at the scheduled time point closest to the patient's surgical procedure end time.

If the patient's pain was not controlled on the protocol recommended pain regimen, the first course of action was to adjust the PCA dosing. Non-steroidal anti-inflammatory medications and/or 5HT3 antagonist anti-emetics were only utilized as RESCUE medications if needed.

End of Treatment Assessments. The patient had reached end of treatment status twenty-four hours (24) after they could tolerate solid foods, were discharged from the hospital, seven (7) days had elapsed, or the patient had been withdrawn or terminated early for any reason from the study.

### Example 3

### Study Procedures

The following procedures were performed for each patient as specified below:

Physical Exam/Vital Signs (Screening and End of Treatment or Early Termination Visits)

Laboratory Assessments (Screening and End of Treatment or Early Termination Visits)

Electrocardiogram (ECG)

Pre & Post Dosing Vital Signs

Verbal Numerical Scale/Vomiting Assessment

Presence or Absence of Nasogastric Tube (NGT) or Orogastric Tube (OGT)

Liquid Challenge

Regardless of bowel sounds, the patient was given 30 cc of water by mouth (measured with an oral syringe) by a qualified research designee each time the patient was seen at 7 am, 12 pm, 5 pm, and 10 pm, ±30 minutes, starting with the first scheduled time point the morning following surgery until the patient could tolerate this 30 cc of liquids. Inability to tolerate clear liquids was defined as nausea and/or vomiting within the first 60 minutes of the challenge.

Bowel Sounds Auscultation

Four times daily at 7 am, 12 pm, 5 pm, and 10 pm, ±30 minutes, the patient's abdomen was auscultated with a stethoscope in all four quadrants (1 minute per quadrant for a total of four (4) minutes) by a qualified research designee starting with the closest scheduled time point time immediately post-op. The first indication of any audible sounds through the stethoscope in any quadrant were considered first bowel sounds.

Full Liquids and Solid Food Advancement

The patient's diet was advanced to full liquids (≧500 cc of liquid) at the meal following the patient's tolerance of their first 30 cc of water by mouth. The patient's diet was then advanced to solid foods at the meal following their tolerance of full liquids. Tolerance of full liquids was defined as no clinically significant nausea and no vomiting within the first hour of the conclusion of the full liquids. Once the patient's diet was advanced to solid foods the patient needed to be observed for two consecutive solid food meals prior to making a determination of whether the meal was tolerated or not. If within one hour of the conclusion of the SECOND consecutive solid food meal, the patient did not report any clinically significant nausea and did not vomit then the solid food toleration endpoint was recorded as after the time of the conclusion of the FIRST solid food meal.

Passage of Flatus/Bowel Movement

The first passage of flatus and first post-operative bowel movement were recorded in the patient's diary and then reviewed by a qualified research designee at each of their daily visits with the patient. This information was recorded in the patient's medical record and CRF.

Foley Catheter

The date and time the patient's foley catheter was removed as well as the time to the patient's first micturition post foley catheter removal were recorded.

Discharge Eligibility

Conventional discharge eligibility was defined as tolerating solid foods, having had at least one bowel movement, normal body temperature, and no major complications.

Secondary Endpoint Follow-up and 30-Day Patient Status Evaluation

Secondary efficacy endpoints along with patient status and ongoing adverse event evaluation were evaluated 30 days after the last dose of study drug.

Adverse Events

Prior and Concomitant Medications

### Example 4

### Results for Segmental Colectomy Patients

Compared to placebo, an intent-to-treat analysis provided the following statistical measures of improvement in gastrointestinal recovery after MNTX administration at 0.30 mg/kg of patient body weight for the group segmental colectomy patients described above. The following results were computed comparing the MNTX treated group of segmental colectomy patients to the placebo group of segmental colectomy patients.

Analysis of the data yielded the following statistical measures of improvement in gastrointestinal recovery by MNTX treatment relative to placebo:
Time to tolerance of first full-liquid meal (p=0.05), accelerated by 30 hours.
Time to tolerance of first solid meal (p=0.12), accelerated by 25 hours.
Time to first bowel movement (p=0.01), accelerated by 23 hours.
Time to tolerance of first solid meal and first bowel movement (p=0.06), accelerated by 27 hours.
Time to discharge eligibility (p=0.03), accelerated by 30 hours.
Time to actual discharge (p=0.09), accelerated by 25 hours.

Analysis of gastrointestinal recovery times were performed using Kaplan-Meier time-to-event methods, and the differences between the means of the MNTX treated and placebo treated groups from this analysis are shown. Statistical evaluations were based on a predefined intent-to-treat analysis, using a log-rank 1-sided test. Statistical significance was determined at the 0.05 level, when MNTX treatment was compared to placebo, with no adjustments for multiple comparisons.

### Example 5

### Results for Sigmoidectomy Patients

Results were similarly analyzed comparing the post-operative MNTX treatment of sigmoidectomy patients to placebo sigmoidectomy patients. The results are shown as follows:

Time to first bowel movement (p=0.009), accelerated by 23 hours.

Time to discharge eligibility (p=0.01), accelerated by 40 hours.

Time to actual discharge (p=0.05), accelerated by 30 hours.

Time to first flatus (p=0.021), accelerated by 25 hours.

Time to tolerance of first solid meal (p=0.15), accelerated by 29 hours.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1-44. (canceled)

45. A method of treating a human patient post-operatively after a segmental colectomy, comprising:
initiating a parenteral administration of a quaternary derivative of noroxymorphone to a patient after a segmental colectomy,
wherein the quaternary derivative of noroxymorphone is administered in an amount sufficient to increase the likelihood of shortening the time to a first bowel movement by the patient after the segmental colectomy.

46. A method of treating a human patient post-operatively after a segmental colectomy, comprising:
initiating a parenteral administration of a quaternary derivative of noroxymorphone to a patient after a segmental colectomy,
wherein the quaternary derivative of noroxymorphone is administered in an amount sufficient to increase the likelihood of shortening the time to discharge eligibility of the patient after the segmental colectomy.

47. The method of claim 46, wherein the quaternary derivative of noroxymorphone is administered in an amount sufficient to increase the likelihood of shortening the time to actual discharge of the patient after the segmental colectomy.

48. A method of treating a human patient post-operatively after a segmental colectomy, comprising:
initiating a parenteral administration of a quaternary derivative of noroxymorphone to a patient after a segmental colectomy,
wherein the quaternary derivative of noroxymorphone is administered in an amount sufficient to increase the likelihood of shortening the time to patient ingestion of a first solid meal and a first bowel movement by the patient after the segmental colectomy.

49. A method of treating a human patient post-operatively after a segmental colectomy, comprising:
initiating a parenteral administration of a quaternary derivative of noroxymorphone to a patient after a segmental colectomy,
wherein the quaternary derivative of noroxymorphone is administered in an amount sufficient to increase the likelihood of shortening the times to:
a first bowel movement by the patient after the segmental colectomy;
discharge eligibility of the patient after the segmental colectomy; and,
patient ingestion of a first solid meal and a first bowel movement by the patient after the segmental colectomy.

50. The method of claim 45, wherein the segmental colectomy is a sigmoidectomy.

51. The method of claim 45, wherein the segmental colectomy is a right hemicolectomy, a left hemicolectomy, a transverse colectomy, a colectomy takedown, or a low anterior resection (LAR).

52. The method of claim 45, wherein the segmental colectomy results from a surgical operation that is performed in about two hours or less.

53. The method of claim 45, wherein the quaternary derivative of noroxymorphone is administered by injection.

54. The method of claim 53, wherein the injection is intravenous.

55. The method of claim 45, wherein the administration of the quaternary derivative of noroxymorphone is initiated less than 7 days after surgery.

56. The method of claim 55, wherein the administration of the quaternary derivative of noroxymorphone is initiated less than one day after surgery.

57. The method of claim 56, wherein the administration of the quaternary derivative of noroxymorphone is initiated about 90 minutes after surgery.

58. The method of claim 45, wherein the first bowel movement occurs within 5 days after the first administration of the quaternary derivative of noroxymorphone.

59. The method of claim 58, wherein the first bowel movement occurs within 4 days after the first administration of the quaternary derivative of noroxymorphone.

60. The method of claim 59, wherein the first bowel movement occurs within 3.5 days after the first administration of the quaternary derivative of noroxymorphone.

61. The method of claim 60, wherein the first bowel movement occurs within 3 days after the first administration of the quaternary derivative of noroxymorphone.

62. The method of claim 46, wherein the patient is eligible for discharge within 5 days after the first administration of the quaternary derivative of noroxymorphone.

63. The method of claim 62, wherein the patient is eligible for discharge within 4 days after the first administration of the quaternary derivative of noroxymorphone.

64. The method of claim 48, wherein the patient ingests a first solid meal and has a first bowel movement within 5 days after the first administration of the quaternary derivative of noroxymorphone.

65. The method of claim 64, wherein the patient ingests a first solid meal and has a first bowel movement within 4.5 days after the first administration of the quaternary derivative of noroxymorphone.

66. The method of claim 65, wherein the patient ingests a first solid meal and has a bowel movement within 4 days after the first administration of the quaternary derivative of noroxymorphone.

67. The method of claim 45, wherein the quaternary derivative of noroxymorphone is administered per dose at about 0.05 to 0.45 mg/kg body weight of the patient.

68. The method of claim 67, wherein the quaternary derivative of noroxymorphone is administered between about once per hour and about once per day.

69. The method of claim 68, wherein the quaternary derivative of noroxymorphone is administered about once every six hours.

70. The method of claim 45, wherein the quaternary derivative of noroxymorphone is methylnaltrexone.

71. The method of claim 45, wherein the quaternary derivative of noroxymorphone is administered at a dose that is less than 50% of the dose at which orthostatic hypotension first appears in humans.

72. The method of claim 45, wherein the quaternary derivative of noroxymorphone is administered at a dose that is less than 50% of the dose at which a lowering of mean arterial blood pressure first appears in humans.

73. The method of claim 45, wherein a quaternary derivative of noroxymorphone is administered to the patient orally after a first period of parenteral administration.

74. The method of claim 45, further comprising administering an anti-emetic composition to the patient.

75. The method of claim 45, further comprising administering an anti-microbial agent to the patient.

76. The method of claim 75, wherein the anti-microbial agent is an antibiotic or an anti-viral agent.

77. The method of claim 45, further comprising administering an opioid to the patient.

78. The method of claim 77, wherein a composition comprising a combination of the opioid and the quaternary derivative of noroxymorphone is administered to the patient.

79. The method of claim 45, wherein morphine is being administered to the patient.

80. The method of claim 79, wherein the patient is weaned off morphine during a period of time over which the quaternary derivative of noroxymorphone is administered to the patient.

81. The method of claim 45, wherein the quaternary derivative of noroxymorphone is administered repeatedly over a time period of between 1 and 7 days.

82. A method of treating a human patient post-operatively after a segmental colectomy, comprising:
initiating a parenteral administration of methylnaltrexone to a patient after a segmental colectomy,
wherein the methylnaltrexone is administered in an amount sufficient to increase the likelihood of shortening a time to:
a first bowel movement by the patient after the segmental colectomy;
discharge eligibility of the patient after the segmental colectomy; and/or,
patient ingestion of a first solid meal and a first bowel movement by the patient after the segmental colectomy.

83. The method of claim 82, wherein methylnaltrexone is infused intravenously.

84. The method of claim 83, wherein methylnaltrexone is administered four times per day at a dose of about 0.3 mg/kg patient weight per administration.

85. The method of claim 84, wherein methylnaltrexone is administered over a period of 1 to 7 days.

86. The method of claim 55, wherein the administration of the quaternary derivative of noroxymorphone is initiated 1 day after surgery.

87. The method of claim 55, wherein the administration of the quaternary derivative of noroxymorphone is initiated at day 2, day 3, day 4, or day 5 after surgery.

88. The method of any one of claim 45, wherein the time is shortened by at least 12 hours.

89. The method of claim 88, wherein the time is shortened by at least 24 hours.
